(19) United States
(12) Patent Application Publication
(10) Pub. No.: US 2026/0159534 A1
(43) Pub. Date: Jun. 11, 2026

(54) NOVEL CRYSTAL FORMS OF PEPTIDE BORIC ACID COMPOUND AND PREPARATION METHODS THEREFOR

(71) Applicant: JIANGSU CHUANGTE PHARMACEUTICAL TECHNOLOGY CO., LTD., Yancheng City, Jiangsu Province (CN)

(72) Inventors: Yongqiang ZHU, Yancheng City (CN); Qi CHEN, Yancheng City (CN); Jia LIU, Yancheng City (CN); Cancan LI, Yancheng City (CN); Yang YANG, Yancheng City (CN)

(73) Assignee: JIANGSU CHUANGTE PHARMACEUTICAL TECHNOLOGY CO., LTD., Yancheng City, Jiangsu Province (CN)

(21) Appl. No.: 18/708,427

(22) PCT Filed: Nov. 7, 2022
(86) PCT No.: PCT/CN2022/130193

§ 371 Date: May 8, 2024

(30) Foreign Application Priority Data
Nov. 8, 2021 (CN) 202111310759.6

(51) Int. Cl. C07F 5/04; A61K 38/00

(52) U.S. Cl. CPC C07F 5/04; A61K 38/00

(57) ABSTRACT

A novel crystal forms of a peptide boric acid compound, and in particular, disclosed are three crystal forms: crystal form A, crystal form B and crystal form D, and preparation methods therefor. The three crystal forms are stable in appearance state and stable in storage, and are suitable for preparing pharmaceutical preparations; moreover, the preparation methods are simple, avoid the use of concentration operations, and are high in product clarity; therefore, the process is simple and efficient, large-scale production can be achieved, and the present invention is environmentally-friendly.

### TECHNICAL FIELD

The present invention relates to crystal forms of a pharmaceutical compound, and more specifically to three novel crystal forms A, B, and D of N—((R)-1-(((R)-1-(1,3,6,2-dioxazaborolan-2-yl)-3-methylbutyl)amino)-3-(methylthio)-1-oxoprop-2-yl)-2,5-dichlorobenzamide and methods for preparing the novel crystal forms.

### BACKGROUND ART

Ubiquitin-proteasome pathway (UPP) is the major pathway for the degradation of intracellular protein systems and is involved in many physiologically important cellular processes, including signal transduction, immune responses, unfolded protein responses, and cell cycle progression. This pathway is closely related to the development of cardiovascular and cerebrovascular diseases, cancers, and nervous system degenerative diseases. Proteasome inhibitors have been used for treating disease, and the first proteasome inhibitor was approved for marketing in 2003. Currently, representative proteasome inhibitors used clinically are bortezomib, carfilzomib, and ixazomib. Bortezomib and carfilzomib are administered by intravenous injection, which needs to be performed by professional medical workers, so that the compliance and the quality of life of patients are inevitably reduced; besides, the two drugs exhibit such problems in safety and effectiveness as peripheral neurotoxicity, cardiotoxicity, and drug resistance in clinical application. Ixazomib, although being an oral preparation, has poor clinical effectiveness and needs to be administered in combination with other drugs. Therefore, there is a need to develop a novel oral proteasome inhibitor with high effectiveness and low toxicity.

The patent CN112384519 discloses N—((R)-1-(((R)-1-(1,3,6,2-dioxazaborolan-2-yl)-3-methylbutyl)amino)-3-(methylthio)-1-oxoprop-2-yl)-2,5-dichlorobenzamide as a proteasome inhibitor for the treatment of solid and hematological tumors, and the structure of the compound is shown as formula (I) below.

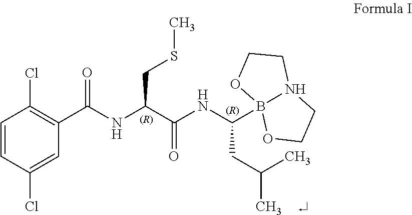

Different crystal forms of the same compound may change the physicochemical properties of the compound, e.g., resulting in different solubility, thermodynamic stability, and density or melting points of the different forms. For a compound with the potential of being developed into a drug, these properties may directly affect the handling or production of the compound as a drug substance and preparation, and such physicochemical properties as stability, solubility, and bioavailability of the preparation may be affected, which may have a significant impact on the efficacy or bioavailability of the active ingredient. Therefore, it is of great significance to obtain a stable crystal form of a compound.

### SUMMARY

The present invention mainly provides novel crystal forms of N—((R)-1-(((R)-1-(1,3,6,2-dioxazaborolan-2-yl)-3-methylbutyl)amino)-3-(methylthio)-1-oxoprop-2-yl)-2,5-dichl orobenzamide and preparation methods therefor, specifically as follows:

Provided is a crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 2θ being ±0.2.

Provided is a method for preparing the crystal form A, which is as follows:
step (1): dissolving an organic base in an organic solvent I and heating to 70-75° C.;
step (2): adding ((R)-1-((R)-2-(2,5-dichlorobenzamido)-3-(methylthio)propionamido)-3-methylbutyl)boric acid/organic solvent I solution dropwise to the solution obtained in step (1) while maintaining the temperature at 70-75° C.;
step (3): after the dropwise addition, cooling to 60° C., stirring for 2 h, cooling to 20-25° C., stirring for 8 h, filtering, and drying to obtain a eutectic solid;
step (4): dissolving the eutectic solid in an organic solvent II, stirring for 2 h at 25-35° C., heating to 55-65° C. and stirring for 2 h with a heating rate of 15° C./h, stirring for 2 h at 25-35° C. with a cooling rate of 15° C./h, and crystallizing;
step (5): filtering and drying to obtain the crystal form A;
wherein, the organic base is selected from diethanolamine, ethanolamine, or ethylenediamine;
the solvent I or II is selected from ethyl acetate, dichloromethane, ketone solvents, ether solvents, alcohol solvents, nitrile solvents, or mixtures thereof;
the volume-to-mass ratio of the solvent I to the organic base is 15-45:1 and is expressed in mL/g;
the volume-to-mass ratio of the solvent II to the eutectic solid is 5-15:1 and is expressed in mL/g;
The volume-to-mass ratio of the organic solvent I solution to ((R)-1-((R)-2-(2,5-dichlorobenzamido)-3-(methylthio)propionamido)-3-methylbutyl)boric acid in the ((R)-1-((R)-2-(2,5-dichlorobenzamido)-3-(methylthio)propionamido)-3-methylbutyl)boric acid/organic solvent I solution is 1-5:1 and is expressed in mL/g.

Provided is a crystal form B of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form B has characteristic peaks at the following diffraction angles 2θ: 8.44, 14.39, 15.08, 16.98, 19.76, 22.17, 22.41, 22.53, and 25.62, the measurement error of 2θ being ±0.2.

Provided is a method for preparing the crystal form B, which is as follows:
step (1): dissolving the crystal form A according to claim 1 in an organic solvent III and stirring at 60° C. for complete dissolving;
step (2), adding a proper amount of a solvent IV dropwise to the solution obtained in step (1), crystallizing, filtering, and drying to obtain the crystal form B;
wherein, the solvent III and the solvent IV are independently selected from dimethylformamide, petroleum ether, ketone solvents, ether solvents, alcohol solvents, nitrile solvents, or the mixture thereof;
the volume-to-mass ratio of the solvent III to the raw material crystal form A is 5-30:1 and is expressed in mL/g;
the volume-to-mass ratio of the solvent IV to the raw material crystal form A is 80-120:1 and is expressed in mL/g.

Provide is a crystal form D of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form D has characteristic peaks at the following diffraction angles 2θ: 6.88, 8.26, 9.15, 11.47, 13.82, 16.61, 18.39, 19.64, and 20.80, the measurement error of 2θ being ±0.2.

Provided is a method for preparing the crystal form D, which comprises the following steps:
step (1): dissolving the crystal form A according to claim 1 in an organic solvent V, stirring at 60° C. for 5 h, and crystallizing;
step (2): filtering and drying to obtain the crystal form D;
wherein, the solvent V is selected from dimethylformamide, petroleum ether, ketone solvents, ether solvents, alcohol solvents, nitrile solvents, or the mixture thereof; further, the volume-to-mass ratio of the solvent V to the raw material crystal form A is 5-15:1 and is expressed in mL/g.

The present invention also provides a pharmaceutical composition, which comprises a pharmaceutically acceptable carrier and one crystal or a combination of two or more crystals selected from the group consisting of:
(a) the crystal form A of the present invention;
(b) the crystal form B of the present invention;
(c) the crystal form D of the present invention.

The crystal form A, the crystal form B, or the crystal form D of the compound of formula (I), N—((R)-1-(((R)-1-(1,3,6,2-dioxazaborolan-2-yl)-3-methylbutyl)amino)-3-(methylthio)-1-oxoprop-2-yl)-2,5-dichlorobenzamide, disclosed in the present invention can be used for preparing a medicament for preventing or treating solid tumors and hematological tumors. The solid tumors include non-small cell lung cancer, small cell lung cancer, lung adenocarcinoma, squamous cell lung cancer, pancreatic cancer, mammary cancer, prostatic cancer, liver cancer, skin cancer, epithelial cell cancer, gastrointestinal stromal tumor, or nasopharyngeal carcinoma. The hematological tumor-related diseases include leukemia, multiple myeloma, mantle cell lymphoma, or histiocytic lymphoma.

The crystal form A, the crystal form B, or the crystal form D of the compound of formula (I) disclosed in the present invention can be administered alone or in combination with other drugs that can effectively treat the diseases for preventing and treating solid tumors and hematological tumor-related diseases.

The present invention also provides a pharmaceutical composition for preventing or treating solid tumors and hematological tumor-related diseases, which comprises a therapeutically effective amount of the crystal form A, the crystal form B, or the crystal form D of the compound of formula (I) disclosed in the present invention and a pharmaceutically acceptable carrier. The pharmaceutical composition may be a common tablet, sustained-release tablet, controlled-release tablet, capsule, granule, pulvis, oral liquid, or injection.

The three crystal forms provided in the present invention have relatively high solubility and good stability, are beneficial to long-term storage and placement of drugs, and are suitable for preparing drug preparations; the preparation methods are simple and feature good repeatability and high yield, the concentration operation is avoided, and the clarity of the product is high; the process is simple and efficient, can realize large-scale production, and is environment-friendly.

### BRIEF DESCRIPTION OF THE DRAWINGS

FIG. 1 shows the X-ray powder diffraction pattern of the white eutectic solid sample obtained in Example 1.

FIG. 2 shows the X-ray powder diffraction pattern of the crystal form A, the product obtained in Example 1.

FIG. 3 shows the DSC pattern of the crystal form A, the product obtained in Example 1.

FIG. 4 shows the infrared spectrum of the crystal form A, the product obtained in Example 1.

FIG. 5 shows the X-ray powder diffraction pattern of the product, the crystal form B, obtained in Example 2.

FIG. 6 shows the DSC pattern of the crystal form B, the product obtained in Example 2.

FIG. 7 shows the infrared spectrum of the crystal form B, the product obtained in Example 2.

FIG. 8 shows the X-ray powder diffraction pattern of the crystal form D, the product obtained in Example 3.

FIG. 9 shows the DSC pattern of the crystal form D, the product obtained in Example 3.

FIG. 10 shows the infrared spectrum of the crystal form D, the product obtained in Example 3.

### DETAILED DESCRIPTION

The present invention is further described below with reference to specific examples according to the general technical knowledge and conventional practice in the art. The following examples are only some of the preferred examples of the present invention and should not be construed as limiting the present invention, and it is apparent to those skilled in the art that several modifications can be made without departing from the scope of the present invention and these modifications shall also fall within the protection scope of the present invention.

### Example 1

### Preparation of Crystal Form A

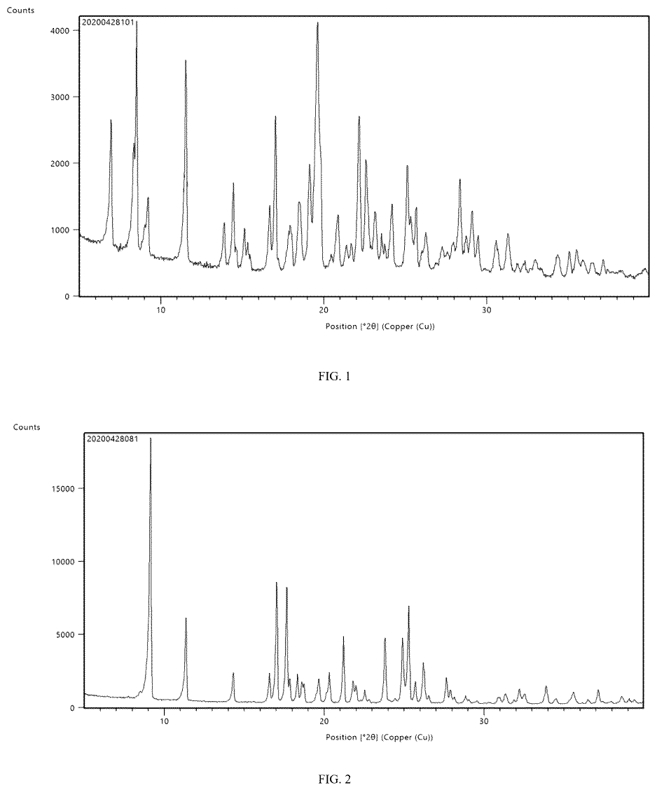

40 g of diethanolamine and 1.20 L of ethyl acetate were added to a reaction vessel, and the system was heated to 70° C. for dissolving. The temperature of the system was maintained at 70° C., and the ((R)-1-((R)-2-(2,5-dichlorobenzamido)-3-(methylthio)propionamido)-3-methylbutyl)boric acid/ethyl acetate solution (dissolving 150 g of ((R)-1-((R)-2-(2,5-dichlorobenzamido)-3-(methylthio) propionamido)-3-methylbutyl)boric acid in 0.3 L of ethyl acetate solution) was added dropwise. After the dropwise addition, the system was cooled to 60° C. and stirred at 60° C. for 2 h, and then the system was cooled to 25° C. and stirred for 8 h while maintaining the temperature at 25° C. The system was filtered, and the filter cake was dried to obtain 147 g of a white eutectic solid, and the X-ray powder diffraction pattern is shown in FIG. 1.

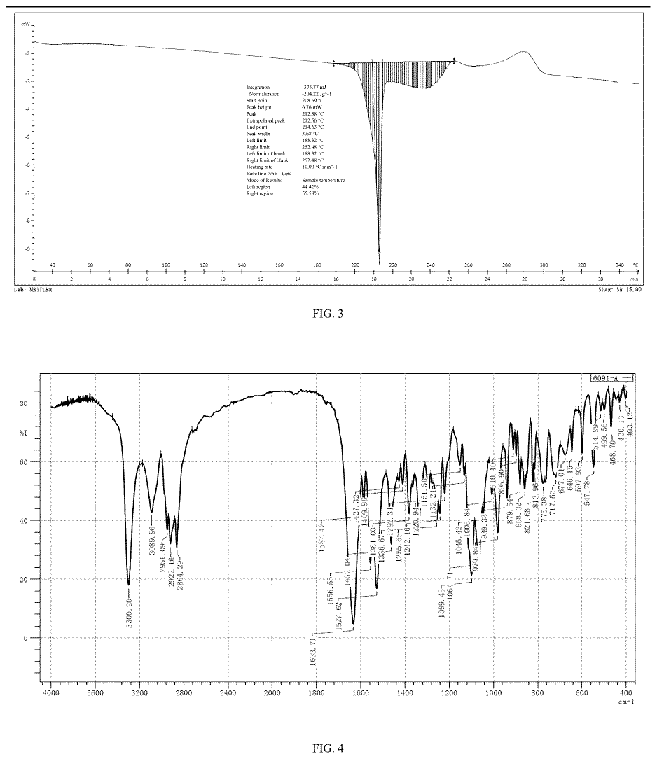

5 g of the eutectic solid sample obtained above and 50 mL of acetonitrile were added to a reaction vessel, and the system was stirred for 2 h at 30° C. Then the system was heated to 60° C. with a heating rate of 15° C./h and slurried for 2 h, and then the system was cooled to 30° C. with a cooling rate of 15° C./h and slurried for 2 h. The system was filtered and dried at 35° C. to obtain 4.8 g of a white solid, namely the crystal form A. The X-ray powder diffraction pattern is shown in FIG. 2, the DSC pattern is shown in FIG. 3, and the infrared spectrum is shown in FIG. 4.

### Example 2

### Preparation of Crystal Form B

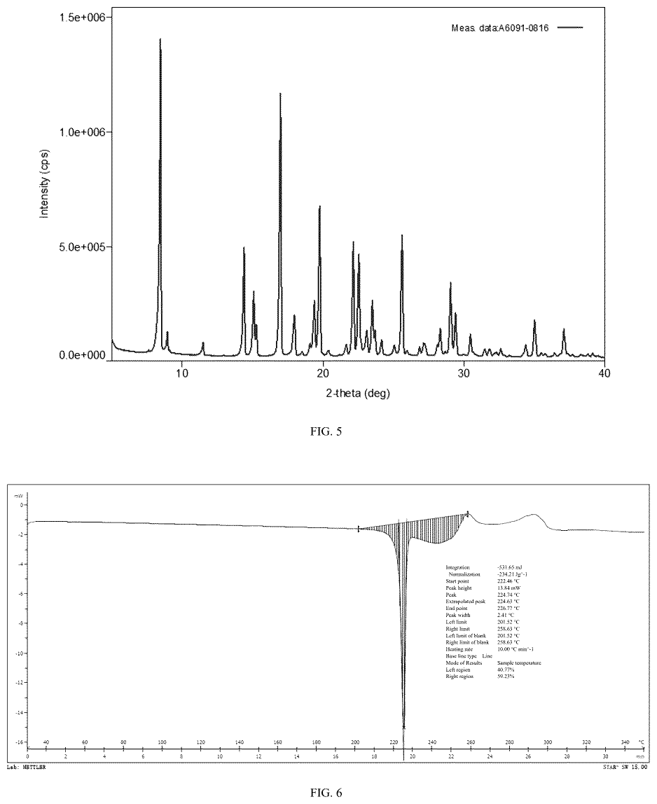

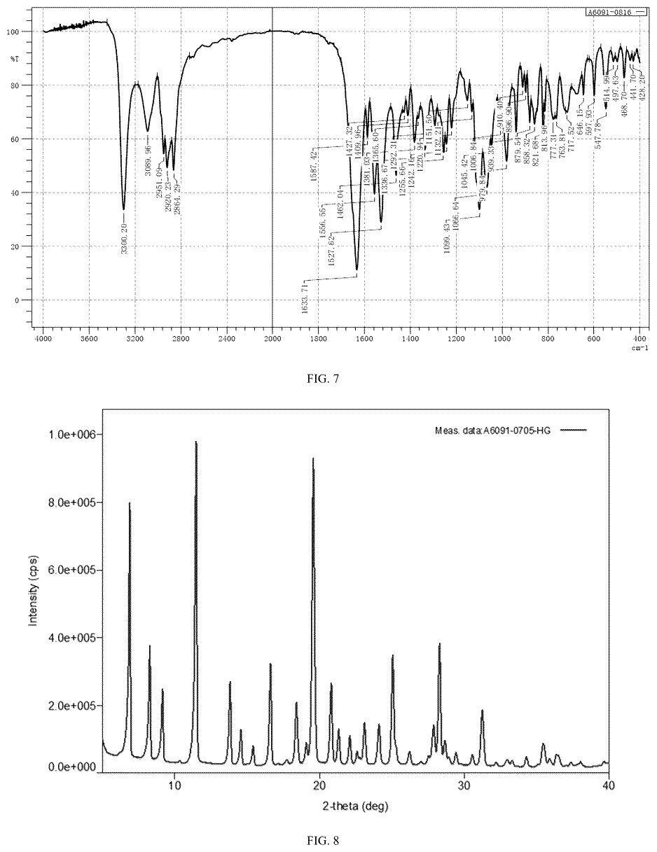

1 g of the crystal form A sample and 20 mL of N,N-dimethylformamide were added to a reaction vessel, and the system was heated to 65° C. for dissolving. 100 mL of petroleum ether was added dropwise, followed by crystallization. The system was dried at 35° C. to obtain 0.72 g of a white solid, namely the crystal form B. The X-ray powder diffraction pattern is shown in FIG. 5, the DSC pattern is shown in FIG. 6, and the infrared spectrum is shown in FIG. 7.

### Example 3

### Preparation of Crystal Form D

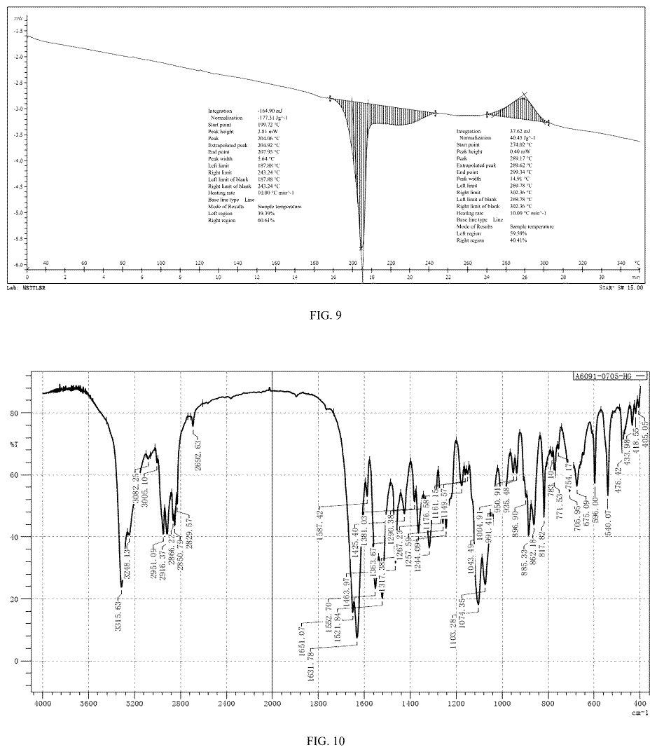

1 g of the crystal form A sample and 10 mL of tetrahydrofuran were added to a reaction vessel, and the system was heated to 60° C. and stirred for 5 h. Then the system was filtered and dried at 25° C. to obtain 0.85 g of a white solid, namely the crystal form D. The X-ray powder diffraction pattern is shown in FIG. 8, the DSC pattern is shown in FIG. 9, and the infrared spectrum is shown in FIG. 10.

### Example 4

Four crystal samples produced, namely the eutectic sample and the crystal form A sample of Example 1, the crystal form B sample of Example 2, and the crystal form D sample of Example 3, were subjected to solubility tests and a kinetic solubility determination tests in ultra-pure water, and the solubility results are shown in Tables 1 and 2. It can be seen that the eutectic and the crystal forms A, B, and D of the compound (I) exhibit excellent solubility property.

TABLE 1
Solubility of four crystal forms of Examples
1, 2, and 3 in ultra-pure water

| Crystal forms | Dissolving | Upper limit | Solubility |
|---|---|---|---|
| of Examples | medium | (μg/mL) | (μg/mL) |
| White eutectic | Ultra-pure | 300 | 310.12 |
| solid of Example 1 | water | | |
| Crystal form A | Ultra-pure | 300 | 318.09 |
| of Example 1 | water | | |
| Crystal form B | Ultra-pure | 300 | 302.80 |
| of Example 2 | water | | |
| Crystal form D | Ultra-pure | 300 | 304.21 |
| of Example 3 | water | | |

TABLE 2
Kinetic solubility of four crystal forms of
Examples 1, 2, and 3 in ultra-pure water

| Crystal forms | Dissolving | Upper limit | Solubility |
|---|---|---|---|
| of Examples | medium | (μg/mL) | (μg/mL) |
| White eutectic | Ultra-pure | 1000 | 948.16 |
| solid of Example 1 | water | | |
| Crystal form A | Ultra-pure | 1000 | 958.47 |
| of Example 1 | water | | |
| Crystal form B | Ultra-pure | 1000 | 1078.59 |
| of Example 2 | water | | |
| Crystal form D | Ultra-pure | 1000 | 902.91 |
| of Example 3 | water | | |

Note: any value near or above the upper limit indicates that the solubility of the compound may reach or exceed the upper limit.

### Example 5

Four crystal samples produced, namely the eutectic sample and the crystal form A sample of Example 1, the crystal form B sample of Example 2, and the crystal form D sample of Example 3, were subjected to hygroscopicity tests at 25° C. under a changing relative humidity of 0-80% RH. It can be seen that the crystal forms A and B of the compound (I) exhibit weaker hygroscopicity compared with the crystal form D and are more suitable for the preparation of solid preparations.

TABLE 3
Results of dynamic vapor sorption determination
of four crystal forms of Examples 1, 2, and 3

| | Weight change in | |
|---|---|---|
| | the range of 0% | |
| Crystal forms of Examples | RH to 80% RH | Hygroscopicity |
| White eutectic solid of Example 1 | 2.1% | Deliquescent |
| Crystal form A of Example 1 | 1.4% | Slightly |
| | | hygroscopic |
| Crystal form B of Example 2 | Less than 0.2% | Nonhygroscopic |
| Crystal form D of Example 3 | 2.4% | Deliquescent |

### Example 6: Stability Tests of Different Crystal Forms of Compound (I)

Four crystal samples produced, namely the eutectic sample and the crystal form A sample of Example 1, the crystal form B sample of Example 2, and the crystal form D sample of Example 3, were each subjected the stability influence factor tests under high temperature (60° C., in the dark), high humidity (relative humidity of 90%±50, in the dark), and illumination (4500 lx±500 lx), and sampling was carried out to on day 0, day 5 day, and day 10 for TIPLC determination, and the results are shown in Table 4 below. During the 10 days of examination, the eutectic sample of Example 1 exhibited significant content change under high humidity, and the eutectic sample was unstable. During the 10 days of examination, the crystal form A, B, and D samples showed no significant content change, the crystal forms didn't change, and the quality was stable.

TABLE 4
Stability results of eutectic sample and crystal form
A sample of Example 1, crystal form B sample of Example
2, and crystal form D sample of Example 3

| | | Crystal forms of Examples | | | |
|---|---|---|---|---|---|
| | | Eutectic | Crystal | Crystal | Crystal |
| | | sample of | form A of | form B of | form D of |
| Conditions | | Example 1 | Example 1 | Example 2 | Example 3 |
| 0 h | | 98.10% | 99.01% | 99.16% | 98.05% |
| 5 d | High | / | 98.97% | 98.92% | 97.68% |
| | temperature | | | | |
| | Illumination | / | 98.61% | 99.22% | 98.07% |
| | High humidity | / | 99.24% | 98.41% | 97.88% |
| 10 d | High | 97.49% | 99.22% | 99.25% | 98.52% |
| | temperature | | | | |
| | Illumination | 98.06% | 98.90% | 99.11% | 98.49% |
| | High humidity | 93.18% | 99.23% | 99.08% | 98.39% |

1. A crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 2θ being ±0.2.

2. The crystal form A according to claim 1, wherein the crystal form A is prepared mainly by the following steps:
step (1): dissolving an organic base in an organic solvent I and heating to 70-75° C.;
step (2): adding ((R)-1-((R)-2-(2,5-dichlorobenzamido)-3-(methylthio)propionamido)-3-methylbutyl)boric acid/organic solvent I solution dropwise to the solution obtained in step (1) while maintaining the temperature at 70-75° C.;
step (3): after the dropwise addition, cooling to 60° C., stirring for 2 h, cooling to 20-25° C., stirring for 8 h, filtering, and drying to obtain a eutectic solid;
step (4): dissolving the eutectic solid in an organic solvent II, stirring for 2 h at 25-35° C., heating to 55-65° C. and stirring for 2 h with a heating rate of 15° C./h, stirring for 2 h at 25-35° C. with a cooling rate of 15° C./h, and crystallizing;
step (5): filtering and drying to obtain the crystal form A;
wherein, the organic base is selected from diethanolamine, ethanolamine, or ethylenediamine;
the solvent I or II is selected from ethyl acetate, dichloromethane, ketone solvents, ether solvents, alcohol solvents, nitrile solvents, or mixtures thereof;
the volume-to-mass ratio of the solvent I to the organic base is 15-45:1 and is expressed in mL/g;
the volume-to-mass ratio of the solvent II to the eutectic solid is 5-15:1 and is expressed in mL/g.

3. A crystal form B of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form B has characteristic peaks at the following diffraction angles 2θ: 8.44, 14.39, 15.08, 16.98, 19.76, 22.17, 22.41, 22.53, and 25.62, the measurement error of 2θ being ±0.2.

4. A method for preparing the crystal form B according to claim 3, wherein the crystal form B is prepared mainly by the following steps:
step (1): dissolving the crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 2θ being ±0.2 in an organic solvent III and stirring at 60° C. for complete dissolving;
step (2), adding a proper amount of a solvent IV dropwise to the solution obtained in step (1), crystallizing, filtering, and drying to obtain the crystal form B;
wherein, the solvent III and the solvent IV are independently selected from dimethylformamide, petroleum ether, ketone solvents, ether solvents, alcohol solvents, nitrile solvents, or the mixture thereof;
the volume-to-mass ratio of the solvent III to the raw material crystal form A is 5-30:1 and is expressed in mL/g;
the volume-to-mass ratio of the solvent IV to the raw material crystal form A is 80-120:1 and is expressed in mL/g.

5. A crystal form D of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form D has characteristic peaks at the following diffraction angles 2θ: 6.88, 8.26, 9.15, 11.47, 13.82, 16.61, 18.39, 19.64, and 20.80, the measurement error of 2θ being ±0.2.

6. The crystal form D according to claim 5, wherein the crystal form D is prepared mainly by the following steps:
step (1): dissolving the crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 20 being ±0.2 in an organic solvent V, stirring at 60° C. for 5 h, and crystallizing;
step (2): filtering and drying to obtain the crystal form D;
wherein, the solvent V is selected from dimethylformamide, petroleum ether, ketone solvents, ether solvents, alcohol solvents, nitrile solvents, or the mixture thereof;
the volume-to-mass ratio of the solvent V to the raw material crystal form A is 5-15:1 and is expressed in mL/g.

7. A pharmaceutical composition, comprising a pharmaceutically acceptable carrier and one crystal or a combination of two or more crystals selected from the group consisting of:
(a) the crystal form A;
(b) the crystal form B;
(c) the crystal form D.

8. A method for preparing the crystal form A according to claim 1, comprising the following steps:
step (1): dissolving an organic base in an organic solvent I and heating to 70-75° C.;
step (2): adding ((R)-1-((R)-2-(2,5-dichlorobenzamido)-3-(methylthio)propionamido)-3-methylbutyl)boric acid/organic solvent I solution dropwise to the solution obtained in step (1) while maintaining the temperature at 70-75° C.;
step (3): after the dropwise addition, cooling to 60° C., stirring for 2 h, cooling to 20-25° C., stirring for 8 h, filtering, and drying to obtain a eutectic solid;
step (4): dissolving the eutectic solid in an organic solvent II, stirring for 2 h at 25-35° C., heating to 55-65° C. and stirring for 2 h with a heating rate of 15° C./h, stirring for 2 h at 25-35° C. with a cooling rate of 15° C./h, and crystallizing;
step (5): filtering and drying to obtain the crystal form A;
wherein, the organic base is selected from diethanolamine, ethanolamine, or ethylenediamine;
the solvent I or II is selected from ethyl acetate, dichloromethane, ketone solvents, ether solvents, alcohol solvents, nitrile solvents, or mixtures thereof;
the volume-to-mass ratio of the solvent I to the organic base is 15-45:1 and is expressed in mL/g;
the volume-to-mass ratio of the solvent II to the eutectic solid is 5-15:1 and is expressed in mL/g.

9. A method for preparing the crystal form B according to claim 3, comprising the following steps:
step (1): dissolving the crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 2θ being ±0.2 in an organic solvent III and stirring at 60° C. for complete dissolving;
step (2), adding a proper amount of a solvent IV dropwise to the solution obtained in step (1), crystallizing, filtering, and drying to obtain the crystal form B;
wherein, the solvent III and the solvent IV are independently selected from dimethylformamide, petroleum ether, ketone solvents, ether solvents, alcohol solvents, nitrile solvents, or the mixture thereof;
the volume-to-mass ratio of the solvent III to the raw material crystal form A is 10-30:1 and is expressed in mL/g;
the volume-to-mass ratio of the solvent IV to the raw material crystal form A is 80-120:1 and is expressed in mL/g.

10. A method for preparing the crystal form D according to claim 5, comprising the following steps:
step (1): dissolving the crystal form A of N—((R)-1-(((R)-1-(1,3,6,2-dioxazaborolan-2-yl)-3-methylbutyl)amino)-3-(methylthio)-1-oxoprop-2-yl)-2,5-dichlorobenzamide, wherein the X-ray powder diffraction pattern of the crystal form A has characteristic peaks at the following diffraction angles 2θ: 9.09, 11.30, 14.27, 17.00, 17.61, 20.28, 23.75, 24.85, and 25.25, the measurement error of 2θ being ±0.2 in an organic solvent V, stirring at 60° C. for 5 h, and crystallizing;
step (2): filtering and drying to obtain the crystal form D;
wherein, the solvent V is selected from dimethylformamide, petroleum ether, ketone solvents, ether solvents, alcohol solvents, nitrile solvents, or the mixture thereof;
the volume-to-mass ratio of the solvent V to the raw material crystal form A is 5-15:1 and is expressed in mL/g.
